(19) United States
(12) Patent Application Publication
(10) Pub. No.: US 2004/0171982 A1
(43) Pub. Date: Sep. 2, 2004

(54) Single use device for transmitting information

(76) Inventor: Marc Danchin, Roquebrune Cap Martin (FR)

Correspondence Address: JACOBSON HOLMAN PLLC, 400 SEVENTH STREET N.W., SUITE 600, WASHINGTON, DC, 20004, US

(21) Appl. No.: 10/686,534

(22) Filed: Oct. 16, 2003

(30) Foreign Application Priority Data
Oct. 16, 2002 (FR) 02 12877

(51) Int. Cl. A61M 31/00

(52) U.S. Cl. 604/65

(57) ABSTRACT

This invention relates to a single use device for transmitting information, allowing either identification of a circulating product, or management of the dispensing/aspiration of a product, by means of a product dispensing machine, in a patient, characterized in that it integrates a transponder emitting said information by radio-frequencies.

The device is advantageously either a medical tool or a tube connecting said machine to a patient.

### FIELD OF THE INVENTION

The present invention relates to a single-use device for transmitting information, allowing a flow of a product having to be injected into or aspirated from a patient during a surgical operation or when effecting medical care, to be managed and monitored.

### BACKGROUND OF THE INVENTION

In the medical environment, it is indispensable to know for certain the nature of the products which are injected into the patient and the modalities/conditions of injection thereof.

According to the prior art, information on the product injected into or aspirated from the patient, during an operation for example, such as the manufacturing batch number or the name of the product, is known thanks to a procedure sheet which the medical personnel must fill in themselves.

Such information is also inscribed directly on the bag of the product but this still supposes that it is manually transcribed on a support (computer or paper).

In any case, a manipulation is necessary in order to record this information, in order thus to be able to conserve it.

Furthermore, the same machine for dispensing or aspirating a product is used for different types of surgical operations, or for effecting medical care.

Such a machine thus manages a quantity, a flowrate or a determined pressure of product in a manner adapted to the operation. It must therefore be programmed manually before each use.

Consequently, an incorrect programmation may have dramatic consequences on the patient.

The programmation of the product dispensing or aspiration machine should therefore be secured so as to allow only the setting adapted to the type of operation underway.

It would be particularly advantageous to provide a device allowing automatic registration of information relative to the product, as well as automatic setting of the product dispensing or aspiration machine.

### SUMMARY OF THE INVENTION

The object of the present invention is in the form of a single-use device for transmitting information, allowing either the identification of a circulating product or the management of the dispensing/aspiration of a product, by means of a machine for dispensing or aspirating a product, in a patient, characterized in that it incorporates a transponder emitting said information by radio-frequencies.

The device is advantageously either a tool for medical, and particularly surgical, use, or a tube connecting said product dispensing machine to a patient, said product being a fluid, for example a gas.

In order to secure the mode of operation of the product dispensing or aspiration machine, the device according to the invention, when it is used, transmits said information to a radio-frequency receiver.

The device advantageously also incorporates a sensor measuring, for example, the pressure, temperature, volume or flowrate of the product circulating therein.

Said information is advantageously transmitted, via the radio-frequency receiver, to an electronic manager of the machine which may thus modify, without manual intervention, the settings of the product dispensing or aspiration machine.

In order to conserve a support of the different information transmitted, the latter is sent by computer link to an outside computer which can thus edit said information on a printer.

### BRIEF DESCRIPTION OF THE DRAWINGS

The invention will be more readily understood on reading the following description relating to an illustrative and in no way limiting form of embodiment, with reference to the accompanying drawings, in which:

FIG. 1 schematically shows a plan view of a device in the form of a tube in direct connection with a product dispensing or aspiration machine.

FIG. 2 schematically shows a plan view of two devices in the form of tubes in direct connection with the same product dispensing or aspiration machine, but with different applications.

FIG. 3 schematically shows a plan view of a device in the form of a single use tool in indirect connection with a product dispensing or aspiration machine.

### DESCRIPTION OF PREFERRED EMBODIMENT

The object of the present invention is a single use device adapted to be used in a medical, and more particularly surgical, environment.

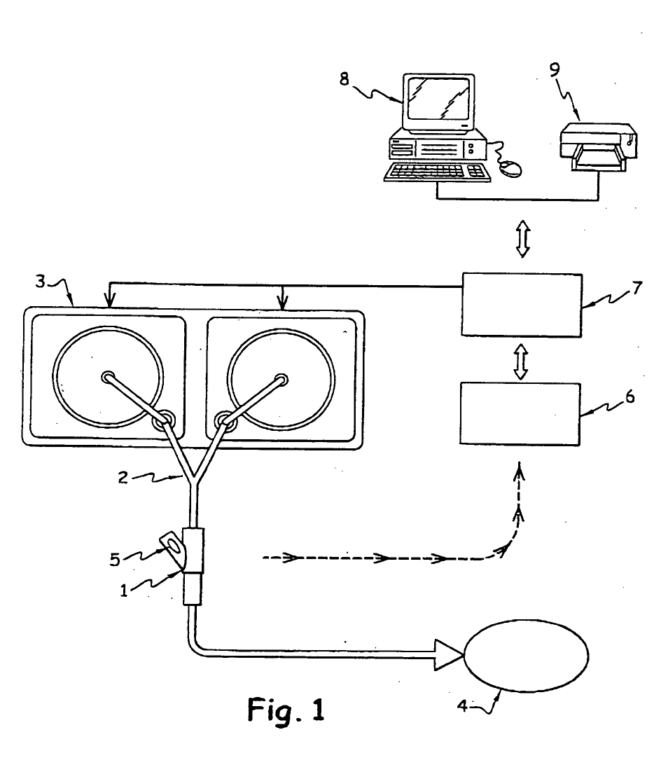

Referring now to the drawings, FIG. 1 shows a tube 1 intended to be incorporated in a tubular assembly 2 allowing the passage of a product dispensed by a product dispensing machine 3.

The tube 1 is intended for single use and must be replaced each time the product dispensing machine 3 is used.

The tubular assembly 2 connects the product dispensing machine 3 to the operative cavity 4.

The patient, or operative cavity 4 receives the product by means of the tubular assembly 2 and the tube 1.

The product dispensing machine 3 makes it possible to set the parameters associated with the product injected, such as its flowrate or its pressure.

The tube 1 incorporates a system of identification by radio-frequencies, i.e. a transponder 5.

The transponder 5 is in any form known per se.

The transponder 5 is for example an electronic chip integrated in a glass tube, a component of plastics material, a label, etc. . . .

During manufacture of the transponder 5 and its integration in the tube 1, data are pre-recorded in the transponder 5.

Said pre-recorded data relate for example to the quantity and to the characteristics of the product having to be used with the tube 1. In effect, a single tube 1 is associated with each bag of product.

The pre-recorded data also concern the function of the product associated with the tube 1, the name of the manufacturer, the manufacturing batch number, time-stamping information, i.e. the date of manufacture and expiry date.

Moreover, the pre-recorded data relate to data of settings specific to the product dispensing machine 3 or other equipment.

The pre-recorded data are transmitted by radio-frequencies to a radio-frequency receiver 6 of type known per se.

The transmission of the pre-recorded data is therefore effected without contact and without electrical link by wire.

The receiver 6 is either integrated directly in the product dispensing machine 3 or integrated in an ancillary equipment (not shown here).

The receiver 6 translates the pre-recorded data received and transmits information concerning settings to the electronic manager 7 of the product dispensing machine 3.

The electronic manager 7 therefore sets the product dispensing machine 3 as a function of the information received.

The information concerning the settings of the product dispensing machine 3 effected by the electronic manager 7 is transmitted by computer link of RS232 or USB type to an outside computer 8 or to a printer 9.

The information emitted by the receiver 6 may also be directly transmitted to the computer 8 or to the printer 9.

The process carried out thus makes it possible to record the pre-recorded data on a data-processing support, to display them on a screen, or to edit them on a paper support.

It is therefore easy to trace the product and said machine 3 is set automatically without outside intervention.

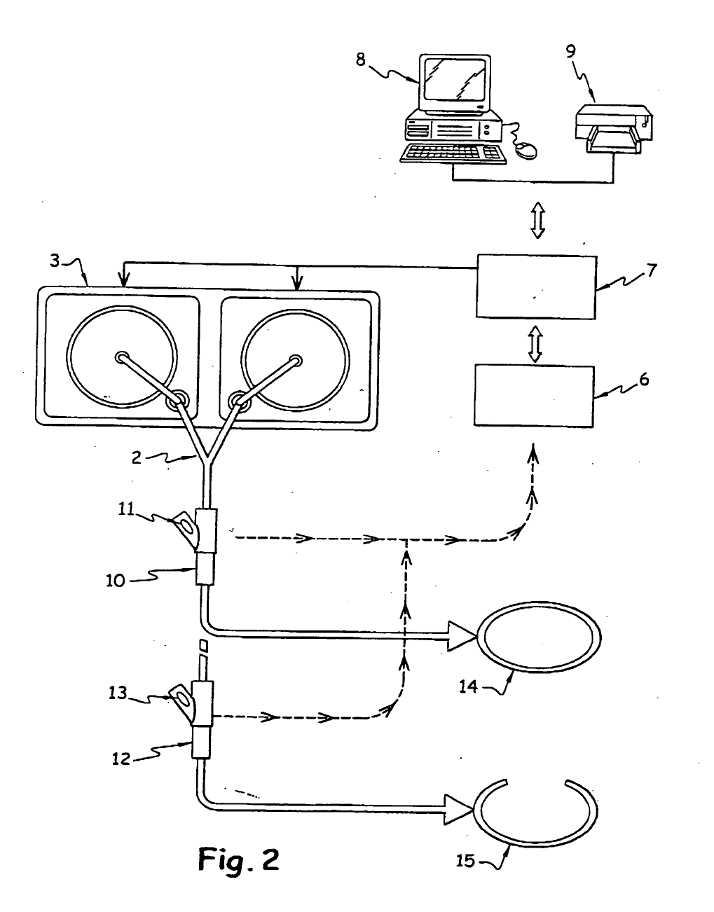

FIG. 2 shows a schematic view of a tubular assembly 2 connected to a product dispensing machine 3 and to an operative cavity 14, 15.

The tube 10 integrating the transponder 11 allows the tubular assembly 2 to function under the conditions required in the operative cavity 14.

The operative cavity 15, different from the operative cavity 14, functions with the same tubular assembly 2 but with the tube 12 integrating the transponder 13.

Tubes 10 and 12 function in the same way as tube 1.

They differ by their pre-recorded data allowing the product dispensing machine 3 to function in two different setting modes.

The receiver 6, the electronic manager 7 as well as the outside computer 8 and printer 9 are of the same type as those of FIG. 1.

The pre-recorded data in the tube 10 and 12 are transmitted by radio-frequencies to the receiver 6 which then transmits information to the electronic manager 7 which, as a function of this information received, adapts the mode of operation of the product dispensing machine 3.

In this way, the product dispensing machine 3 may serve for several types of surgery without said machine 3 requiring to be manually set beforehand.

The machine adapts its functioning and in particular the pressure of the product dispensed, as a function of the tube 10, 12 connected to the tubular assembly 2.

The device according to the invention may, for example, also adapt the flowrate of the product as a function of the type of tube.

For example, with the same product dispensing machine, the operative cavity may be subjected equally well to endoscopic surgery which necessitates dispensing product at a pressure included between 0 and 350 mbars, and to open cavity surgery which requires a pressure of the dispensed product of 1500 mbars.

The device according to the invention thus makes it possible to set, secure and validate the mode of operation of the product dispensing machine as a function of the type of surgery practised in the operative cavity and of the type of tube used.

The tubes 10 and 12 may also advantageously integrate a sensor (not shown in the Figure), of type known per se, making it possible to read in real time or to record parameters relative to the product circulating in the tubular assembly 2 and the product dispensing machine 3.

The parameters of the circulating product are for example the values of the temperature, pressure and flowrate of the product.

These parameters are then transmitted to the transponder 11, 13 which sends them to the receiver 6.

The receiver 6 may then send information to the electronic manager 7 which modifies the setting and mode of operation of the product dispensing machine 3, if certain critical or limiting values are attained, and/or transmit the values of the parameters to the outside computer 8 so that they are recorded and displayed on a screen, and to a printer 9 so that they are edited.

The device according to the invention may equally well be used for surgical operations and for systems for dispensing liquids or medicines.

For example, in the domain of nutrition, perfusion or transfusion, single use tubes according to the invention may retranscribe information relative to the product, in particular the name of the manufacturer, the batch number, the date of manufacture and the expiry date.

The same device according to the invention may also determine whether there has been an abnormal dispensing of the product, by the presence of a flowrate, pressure or temperature sensor.

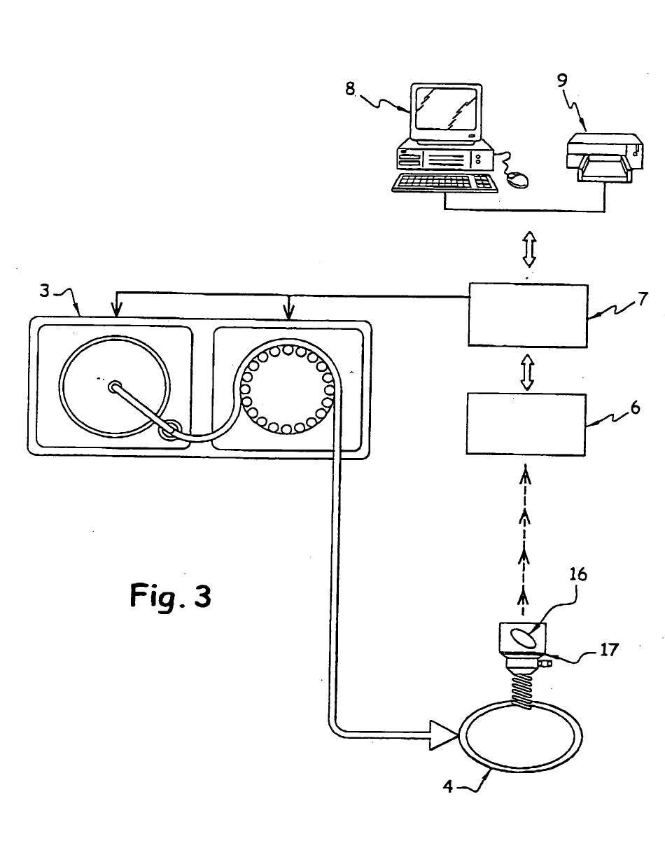

FIG. 3 represents another possible use of the device according to the invention.

The product dispensing machine 3, the receiver 6, the electronic manager 7 as well as the outside computer 8 and printer 9, are of the same type as those of FIG. 1.

The transponder 16 is integrated in a single use tool 17, not connected by a tubular assembly to the product dispensing machine 3.

The tool is, for example, a trocar or a cannula for surgical use.

The tool 17 functions in accordance with the same principle as the devices with tube described above, i.e. pre-recorded information is integrated in the transponder 16 which transmits it to the receiver 6, this then allowing the settings of the product dispensing or aspiration machine 3 to be modified as a function of the nature of that information.

The transponder 16 may also receive information from the product dispensing machine 3 indicating thereto defects or anomalies of said machine 3.

The tool 17 presents a sensor (not shown) of known type.

The sensor makes it possible to measure the flowrate, the pressure or the temperature of the product or fluid circulating in the tool 17.

In this way, the setting of the product dispensing machine 3 may be modified as a function of the value of the parameters measured by the sensor of the tool 17.

The device according to the invention shown in FIG. 3 may thus advantageously be used in endoscopic surgery where tools and/or components are introduced in operative cavities.

Consequently, the device according to the invention may transmit the intra-cavity pressure and temperature in real time, with the aid of a sensor.

What is claimed is:

1. A medical device integrating information transmission means allowing management of the parameters of dispensing/aspiration of a product, by means of a machine for dispensing or aspirating a product, in a patient, and a transponder emitting said information by radio-frequency,
wherein said device is used once only,
it allows a circulating product to be identified, and
it consists of a tube connecting said product dispensing or aspiration machine to a patient.

2. A medical device integrating information transmission means allowing management of the parameters of dispensing/aspiration of a product, by means of a product dispensing or aspiration machine, in a patient, and a transponder emitting said information by radio-frequency,
wherein said device is used once only,
it allows a circulating product to be identified, and
it consists of a medical, particularly surgical, tool.

3. The device of claim 1 or 2, wherein said product is a fluid, particularly a gas.

4. The device of one of the preceding Claims, wherein the transponder integrates a sensor for determining in real time or for recording parameters relative to said circulating product, particularly the pressure, temperature or flowrate of said circulating product.

5. An assembly for the transmission of information comprising the medical device of one of the preceding Claims, wherein it further comprises a radio-frequency receiver for the transmission of information.

6. The assembly of claim 5, wherein it comprises an electronic manager for said machine to which is transmitted said information coming from the radio-frequency receiver and which modifies the setting of the mode of operation of said machine as a function of the information received.

7. The assembly of claim 5 or 6, wherein it comprises an outside computer to which the information emitted by the transponder is sent by computer link after it has been received by the receiver.

8. The assembly of one of claims 5 to 7, wherein it comprises a printer on which the information emitted by the transponder is retranscribed.
